(19) United States
(12) Patent Application Publication
(10) Pub. No.: US 2008/0254496 A1
(43) Pub. Date: Oct. 16, 2008

(54) Methods for Detection of Fungal Disease

(76) Inventor: Jeffrey R. Shuster, Chapel Hill, NC (US)

Correspondence Address: PASSE' INTELLECTUAL PROPERTY, LLC, 1717 BRASSFIELD RD., RALEIGH, NC, 27614, US

(21) Appl. No.: 12/088,107

(22) PCT Filed: Sep. 26, 2006
(86) PCT No.: PCT/US2006/037607

§ 371 Date: Mar. 26, 2008

Related U.S. Application Data
(60) Provisional application No. 60/720,950, filed on Sep. 27, 2005.

(51) Int. Cl. C12Q 1/26

(52) U.S. Cl. 435/25

(57) ABSTRACT

The present invention describes the use of enzymes having D-arabinitol oxidase activity with the concomitant generation of hydrogen peroxide for the diagnosis of fungal infections of humans and other organisms, and for the detection of fungal organisms that are present on or in surfaces or permeable materials. The present invention also describes methods and kits for the detection of fungi, and the diagnosis of fungal infections based upon D-arabinitol oxidase activity. The present invention also provides a new biochemical activity for gene products encoded by bacterial genes previously identified only with putative function. Activity of these gene products includes the ability to oxidize D-arabinitol with the concomitant generation of hydrogen peroxide.

### BACKGROUND OF THE INVENTION

1. Field of the Invention

The present invention relates generally to a method for the detection of fungal disease. More particularly the present invention relates to a method for detecting the presence of D-arabinitol in a test sample.

2. Description of the Related Art

Blood stream infection of microbial origin is a serious concern for patients and for the healthcare system. The most common fungal blood stream infection associated with hospital stays is invasive candidiasis (Wisplinghoff et al., 2004). Positive identification of invasive candidiasis is currently performed using classical microbiological techniques, which involve assaying for the recovery of live fungal cultures from the blood. The problem with this approach is that the method requires two to three days, has a low success rate in the diagnosis of the disease (Berenguer et al., 1993), and patients currently diagnosed using this method have a poor prognosis with a significant chance of death from the disease (Gudlaugsson et al., 2003). Many pathogenic fungi are tissue-invasive and adherent to specific endo- or epithelial tissues, making routine blood microbiological culture methods for these organisms insensitive and unreliable even as the disease progresses. Accordingly, there is a need for an earlier and more reliable detection method for pathogenic fungal infections in order to rapidly intervene with the appropriate treatment, to increase the chance for patient survival, and to reduce the overall intervention costs.

To address the historically poor sensitivity in the diagnosis of fungal infections, the field has been working on the prospect of blood-based surrogate markers. One such test is the β-glucan test (commercially Fungitell™, previously Glucatell, Associates of Cape Cod, (Odabasi et al., 2004)). This test, approved by the United States Food and Drug Administration (FDA) in May, 2004, involves pre-treatment of patient serum, followed by the activation of a modified Limulus amebocyte proteolytic cascade pathway to generate a kinetically monitored chromogenic response via the cleavage of para-nitroaniline peptide substrate. The assay requires a number of specialty reagents and an experienced technician trained in the assay methodology.

Additional methods being investigated for use in the field of fungal diagnostics are the polymerase chain reaction (PCR), and DNA/RNA detection using microarrays. However, PCR diagnostic tests may suffer some of the same biological limitations as do culture methods. Specifically, fungal organisms may be localized in an adherent manner, and thus the fungal DNA may not be circulating where it can be readily analyzed.

Unlike other targets of diagnostic products, D-arabinitol (DA) is a soluble fungal metabolite which is not limited only to the site of infection. Once outside of the producing fungus, it circulates and is detectable in body fluids such as blood and urine. DA was first described as a useful diagnostic of infection by Candida albicans in 1979 (Kiehn et al., 1979), and work with this compound has continued thereafter (Hui et al., 2004). Although Candida albicans is the most common causal agent for candidiasis, other Candida species have been investigated, and many of them also produce this signature metabolite (Bernard, 1981).

The advancement of using DA as a diagnostic continued from gas-chromatography detection (GC) (de Repentigny et al., 1983; Wells et al., 1983), to stereo-specific analysis (Wong and Brauer, 1988; Wong and Castellanos, 1989), GC-mass spectroscopy (GC/MS) (Larsson et al., 1994; Lehtonen et al., 1996), and also by enzymatic conversion of DA to D-ribulose with the generation of NADH (U.S. Pat. Nos., 5,451,517, 5,766,874, 6,280,988, 6,287,833, 6,426,204) (Switchenko et al., 1994; Yeo et al., 2000). In the latter case, the NADH was measured fluorometrically (Yeo et al., 2000), or in coupled reaction with diaphorase (Switchenko et al., 1994). In the above mentioned reports, the levels of DA have been measured in both serum and in urine, and elevated levels have been correlated with candidiasis in clinical samples. Early work around the use of DA as a diagnostic metabolite has been aimed at determining the levels of DA in clinical samples and correlating it with normal controls vs. infected individuals, resolving the D-enantiomer from the endogenous L-enantiomer, and determining the effects of kidney function on the accumulation/clearance of DA in serum and urine.

### SUMMARY OF THE INVENTION

It is an object of the present invention to overcome the limitations and problems inherent with the current methods of detecting fungal disease and the presence of fungi. It is an object of this invention to produce new and novel compositions which aid in the detection of certain fungi specifically D-arabinitol producing fungi. It is further an object of the invention to introduce a novel method of producing compositions useful in the detection of D-arabinitol in the presence of oxygen.

In another aspect, detection of DA is determined by the enzymatic production of hydrogen peroxide and detection of the hydrogen peroxide thus produced. In another aspect, the level of DA in the sample is determined by the extent of hydrogen peroxide produced and measured as compared to a standard. In another aspect, the level of DA in a test sample is used as an indirect indication of the extent of fungal infection. In another aspect, sugar alcohol oxidase enzymes are mutated and/or modified to have improved activity.

One aspect of the invention provides a method of detecting the presence of D-arabinitol, which was produced by a fungus, in a test sample comprising:
a. obtaining a test sample;
b. selecting a predetermined amount of an enzyme having D-arabinitol oxidase activity;
c. contacting the test sample with the enzyme in the presence of oxygen;
d. measuring the amount of hydrogen peroxide produced by the reaction of the test sample and the enzyme; and
e. determining if the amount of hydrogen peroxide produced is sufficient to indicate the presence of D-arabinitol produced by a fungus.

Another aspect of the invention provides a kit of parts suitable for the detection of the presence of a fungus by the detection of the amount of D-arabinitol in a test sample comprising: an enzyme having D-arabinitol oxidase activity capable of oxidizing D-arabinitol in the presence of oxygen to produce hydrogen peroxide and a means for detecting the amount of hydrogen peroxide produced by the reaction of D-arabinitol and the enzyme.

The invention also provides a method of producing an enzyme which preferentially oxidizes D-arabinitol in the presence of oxygen to produce hydrogen peroxide over oxidizing other sugar alcohols comprising:
a) producing at least one mutation in a first gene encoding a first enzyme having D-arabinitol oxidase activity to produce a second gene encoding a second enzyme having D-arabinitol oxidase activity;
b) measuring the enzyme activity, produced by the second enzyme, to oxidize D-arabinitol versus its ability to oxidize at least one other sugar alcohol;
c) calculating the ratio of the D-arabinitol oxidase activity to the oxidase activity with at least one other sugar alcohol for the second enzyme;
d) comparing the calculated ratio for the second enzyme to the same ratio for the first enzyme;
e) selecting the second enzyme if the mutant enzyme has a higher ratio than the first enzyme.

### BRIEF DESCRIPTION OF THE FIGURES

FIG. 1. Enzymatic reaction of a D-arabinitol oxidase.

FIG. 2. A calorimetric method for the detection of hydrogen peroxide.

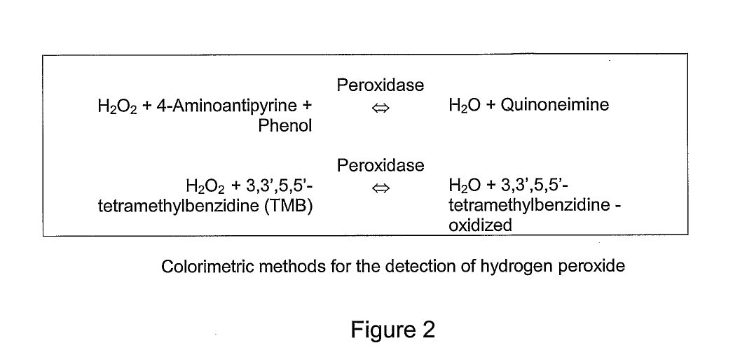

FIG. 3. Multiple sequence alignment of known sugar alcohol oxidase enzymes including bacterial sugar alcohol oxidases previously identified only as a “putative oxidases.”

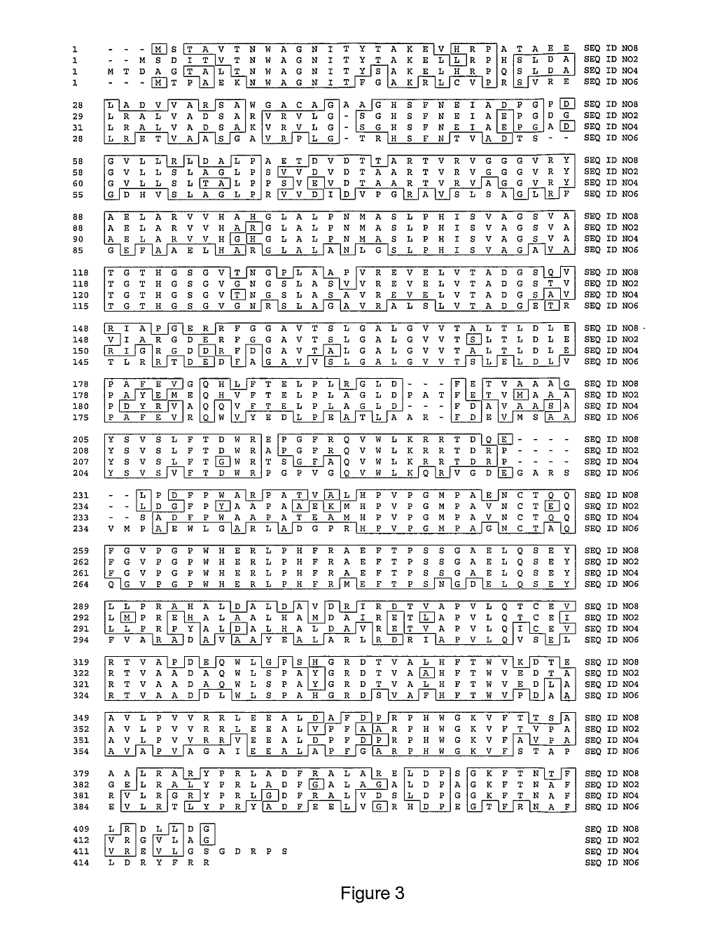

### DETAILED DESCRIPTION OF THE INVENTION

The present invention provides methods for detecting a fungal metabolite, D-arabinitol, as a diagnostic for the presence of a fungus or a fungal infection. Unlike other described methods for the detection of this compound as diagnostic for the presence of fungal organisms, the method described in this invention utilizes a class of oxidoreductase enzymes which react with sugar alcohol (including D-arabinitol) as substrate and oxygen as acceptor (EC 1.1.3) to produce hydrogen peroxide, H₂O₂, as a product. The present invention also describes novel enzymes having D-arabinitol oxidase activity and methods of making them encoded by bacterial genes that had not previously been identified as having D-arabinitol oxidase activity. The activity of these enzymes were determined by the inventors to have the ability to oxidize D-arabinitol and in one embodiment preferentially, with the concomitant generation of hydrogen peroxide.

The present invention also describes methods for the generation of sugar alcohol oxidase enzymes with altered D-arabinitol activity by mutagenizing the gene sequence for known or putative sugar alcohol oxidase enzymes to produce an oxidase enzyme that is selective for D-arabinitol and/or shows high activity against D-arabinitol when compared to known enzymes.

Unless otherwise indicated, the following terms are intended to have the following meanings in interpreting the present invention.

As used herein, the term “coding region” refers to a portion of a nucleic acid sequence, either DNA or RNA, whose particular sequence order of nucleotides encodes a protein. Differences in a nucleotide sequence that do not result in a change in the translated protein sequence are recognized to be the same coding region. It is recognized that a coding region may or may not contain one or more intron sequences which are non-coding sequences that are removed prior to the translation of nucleic acid sequence into protein.

As used herein, the term “coding sequence” refers to a portion of a nucleic acid sequence, either DNA or RNA, whose particular sequence order of nucleotides encodes a protein. Differences in a nucleotide sequence that do not result in a change in the translated protein sequence are recognized to be the same coding sequence.

As used herein, the term DA means D-arabinitol, also known as D-arabitol, D-arabinol, and D-lyxitol.

As used herein, the terms “DAOx” and “D-arabinitol oxidase” and “DA oxidase” each mean a sugar alcohol oxidase that is capable of the interconversion of D-arabinitol and oxygen to D-arabinose and hydrogen peroxide.

As used herein, the term “DNA” means deoxyribonucleic acid.

As used herein, the terms “gene” and “gene sequence”, each refer to heritable units of DNA or RNA. The gene or gene sequence may include regulatory sequences, control sequences and/or intron sequences. It should be recognized that small differences in a nucleotide sequence for the same gene can exist between different strains without altering the identity of the gene.

As used herein, the terms “gene product”, “protein”, and “polypeptide” and herein used interchangeably.

As used herein, the term H₂O₂ means hydrogen peroxide.

As used herein, the term “His-Tag” refers to an encoded polypeptide consisting of multiple histidine amino acids used to assist in protein purification.

As used herein, the term “mutation” refers to an alteration of a gene, gene sequence, coding sequence, or coding region, either naturally or artificially, changing the sequence of nucleotides comprising said gene, gene sequence, coding sequence, or coding region. The change in the base sequence may be of several different types, including changes of one or more bases for different bases, deletions, and/or insertions.

As used herein, the term “Ni” refers to nickel.

As used herein, the term “Ni-NTA” refers to nickel sepharose.

As used herein, the term “PCR” means polymerase chain reaction.

As used herein, the “percent (%) sequence identity” between two polynucleotide or two polypeptide sequences is determined according to the BLAST program (Basic Local Alignment Search Tool; (Altschul, S. F., W. Gish, et al. (1990) J Mol Biol 215: 403-10 (PMID: 2231712)) at the National Center for Biotechnology, or other similar molecular biology homology algorithms well known in the art. It is understood that for the purposes of determining sequence identity when comparing a DNA sequence to an RNA sequence, a thymine nucleotide is equivalent to a uracil nucleotide.

As used herein, the term, “protein” refers to a sequence of amino acids formed into a polypeptide chain of at least ten amino acids in length, comprised of any combination of natural, modified, or chemically synthesized amino acids. It is recognized that a protein may consist of a single linear or circular chain of amino acids, or of polypeptides that are associated by covalent or non-covalent bonds or linkages. Additionally, a protein may contain post-translational chemical modification, either natural or artificial, of any amino acid present in the polypeptide, such as by, but not limited to, conjugation to phosphates, lipids, or carbohydrates.

As used herein, the term “RNA” means ribonucleic acid.

As used herein, the term “test sample” means a sample suspected of containing D-arabinitol (DA) produced by a fungus. A test sample can contain live organism or be a sample that was in contact with a fungus and thus is suspected of containing a fungus product DA. In one aspect, the test sample is derived from urine or blood from a mammal suspected of having a systemic infection with a microbe known to produce DA. In another aspect, the sample is derived from other bodily fluids such as oral, nasal, cerebrospinal, vaginal secretions, or bronchial lavage. In another aspect, the sample is derived from an inanimate object, surface or in permeable materials.

Examples of fungal organisms that produce D-arabinitol include, but are not limited to, Candida albicans, Candida tropicalis, Candida pseudotropicalis, and Candida parapsilosis.

The present invention provides a novel method for the use of enzymes having D-arabinitol oxidase activity with concomitant production of hydrogen peroxide. The enzymes are useful for the diagnosis and detection of the presence of a fungus in humans, other organisms and on inanimate objects.

The methods of the invention are useful for determining the presence or absence of a fungal disease, determining appropriate drug treatment and therapy, monitoring drug efficacy, and staging the severity of disease. In addition, the invention provides methods for the detection of fungal organisms on surfaces or in permeable materials.

In certain embodiments of the invention, methods and kits are provided for the detection of fungi and the diagnosis of fungal infection based upon the detection of D-arabinitol using a D-arabinitol oxidase that generates hydrogen peroxide. A kit will contain an enzyme for reacting with D-arabinitol and a means for detecting hydrogen peroxide produced in the presence of D-arabinitol. The invention also provides nucleotide sequences that encode novel proteins having D-arabinitol oxidase activity for use in detecting DA according to the methods of the invention.

In one aspect, the invention provides methods for identifying and producing gene products having D-arabinitol oxidase activity with generation of hydrogen peroxide. In another aspect, the invention provides nucleic acids that encode proteins having D-arabinitol oxidase activity useful in the methods of the invention. The nucleic acids of the invention include, SEQ ID NO:1, SEQ ID NO:3, SEQ ID NO:5, and SEQ ID NO:7 and the corresponding proteins of the invention include SEQ ID NO:2, SEQ ID NO:4, SEQ ID NO:6, and SEQ ID NO:8.

Also included in the nucleic acids of the invention are those encoding proteins with D-arabinitol oxidase activity and having at least 40% protein sequence identity to SEQ ID NO:2,

preferably 41-49%, and more preferably nucleic acids having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater sequence identity to SEQ ID NO:2.

In another aspect, the invention provides methods for identifying enzymes with higher specificity for oxidizing D-arabinitol preferentially over other sugar alcohols, particularly those sugar alcohols typically found in an infected mammal, including humans. Examples of methods used to modify a gene and thus gene product, include but are not limited to, PCR mediated mutagenesis (error prone PCR), gene shuffling technology, and random mutagenesis.

In one embodiment of the invention, nucleic acids encoding proteins with sugar alcohol oxidase activity are altered to produce variations in the protein coding sequence using methods known to those skilled in the art. These variant proteins are tested for altered activity and/or specificity toward sugar alcohols. Variant proteins with higher specificity toward D-arabinitol than other sugar alcohols and/or higher activity toward D-arabinitol than previously known enzymes are useful in the methods of the invention. In one embodiment, nucleic acids are amplified using error prone polymerase chain reaction (PCR). The PCR products are cloned into expression vectors to produce the protein product.

In another embodiment, a DAOx variant protein having improved activity and/or improved specificity is achieved by random insertion/deletion mutagenesis using a method such as that described in Murakami et al. (2002). Other methods of random integration mutagenesis are known to those skilled in the art.

In another embodiment, a DAOx variant protein having improved activity and/or improved specificity is achieved using random mutagenesis by exposing the DNA encoding the DAOx protein or an organism containing the DNA encoding the DAOx protein to mutagenic agents including but not limited to ultraviolet radiation, ethylmethane sulphonate or other means of mutagenesis known to those skilled in the art. In yet another embodiment the mutant is produced by site directed mutagenesis.

The nucleic acids of the invention can be cloned into a vector for propagation, and for protein expression in a suitable host. The protein product is assayed for the ability to generate H₂O₂ in the presence of DA by methods known to those skilled in the art. This result can be compared to the ability to generate hydrogen peroxide in known enzymes. The nucleic acids of the invention are expressed in an appropriate host organism such as E. coli, yeast, baculovirus, or other expression systems known to those skilled in the art. Crude cellular extracts, enriched extracts (i.e. partially purified proteins), or purified protein are tested for the ability to oxidize DA and produce hydrogen peroxide. Crude cellular extracts, enriched extracts (i.e. partially purified proteins), or purified protein are also tested against a panel of other sugar alcohols such as, but not limited to, arabinitol, xylitol, sorbitol, mannitol, ribitol, galactitol, and i-erythritol in both D- and L-forms as available, and the level of hydrogen peroxide is determined. Nucleic acids that encode for proteins with relatively increased activity against DA and reduced activity against other sugar alcohols are useful in the methods of the invention.

In one embodiment, the nucleic acids are cloned into expression vectors to produce the protein product. Purification of the protein is performed using methods well known to those skilled in the art. In another embodiment, genes are cloned into expression vectors which contain an additional sequence which is fused to the protein at the N-terminus or C-terminus to aide in protein purification. In one embodiment, polyhistidine tags (His-tag) are incorporated into the protein and the protein is purified using a Ni-NTA resin. Other protein affinity tagging methods can be used to aid in protein purification and are known to those skilled in the art.

The amount of hydrogen peroxide generated by a D-arabinitol oxidase in the presence of DA is measured by methods known to those skilled in the art. In one embodiment, hydrogen peroxide is measured using horseradish peroxidase and an oxidation sensitive dye such as 3,3′,5,5′-tetramethylbenzidine hydrochloride (TMB), and measuring the degree of color development spectrophotometrically. In another embodiment, hydrogen peroxide is measured using horseradish peroxidase, phenol, and a oxidation sensitive dye such as 4-aminoantipyrine, to generate quinoneimine, a red colored dye, and measuring the degree of color development spectrophotometrically. In another embodiment, hydrogen peroxide is detected by other colorimetric means using, for example, Amplex Red Reagent or Amplex Ultrared Reagent (Molecular Probes, Inc.). In another embodiment, hydrogen peroxide is detected electrochemically using methods known to those skilled in the art, such as an amperometric immunosensor (Paul Scherrer Institute) or peroxidase redox polymer wired enzyme electrode kit (Bioanalytical Systems, Inc.).

The quantitative amount of hydrogen peroxide generated is used to determine the amount of DA in the original sample by comparison to a standard curve prepared by adding known amounts of DA to a DAOx catalyzed reaction and measuring the production of hydrogen peroxide under specific controlled conditions. In another embodiment, the electron products of the DAOx catalyzed reaction are utilized to determine and correlate the amount of DA in the samples using methods well known to practitioners in the art.

In another embodiment, the invention provides methods to detect D-arabinitol in liquids, on solids, in body fluids, secretions, tissues comprising or the like by contacting them with a D-arabinitol oxidase and determining the quantitative amount of hydrogen peroxide generated whereby the hydrogen peroxide is detected by calorimetric means as previously described.

In another embodiment, the invention provides methods for diagnosing whether an organism has a fungal infection, has been in contact with a fungus or has a fungal colonization by determining the presence, absence, or level of D-arabinitol using a D-arabinitol oxidase which has the ability to oxidize D-arabinitol with the generation of hydrogen peroxide.

In another embodiment, the invention provides methods for producing a kit for the detection of fungal infection, or fungal presence, containing a D-arabinitol oxidase which has the ability to oxidize D-arabinitol with the generation of hydrogen peroxide whereby the kit detects hydrogen peroxide in a liquid matrix.

In another embodiment, the invention provides methods for producing a kit for the detection of fungal infection, or fungal presence containing, a D-arabinitol oxidase which has the ability to oxidize D-arabinitol with the generation of hydrogen peroxide whereby the kit detects hydrogen peroxide on a solid matrix such as a test strip, or an electrochemical grid, or an electrode.

In one embodiment, the kit provides D-arabinitol oxidase enzyme deposited on a test strip. In another embodiment, the kit provides D-arabinitol oxidase enzyme in a solution. A liquid test sample containing D-arabinitol is applied to the test strip thereby producing hydrogen peroxide in proportion to the amount of D-arabinitol in the sample. In one embodiment, the kit provides an indicator dye is oxidized producing a specific color indicating that D-arabinitol was present in the test sample. The amount of color is proportional to the amount of hydrogen peroxide produced during the D-arabinitol oxidase reaction. In another embodiment, the kit provides for detection of hydrogen peroxide via a colorimetric reaction is measured spectrophotometrically and is compared to a standard curve to quantify the level of D-arabinitol in the original sample. In another embodiment, the kit provides for detection of hydrogen peroxide via an electrochemical reaction that is measured electrically and is compared to standards to quantify the level of D-arabinitol in the original sample.

In another embodiment, the invention provides a medical device for the detection of fungal infection, or fungal presence containing, a D-arabinitol oxidase which has the ability to oxidize D-arabinitol with the generation of hydrogen peroxide whereby the medical device detects hydrogen peroxide using calorimetric means. One such medical device consists of a reagent strip containing a solid surface, a DAOx protein, with additional reagents to generate a color when hydrogen peroxide is produced, and an associated reflectometer instrument. A liquid sample to be tested for the presence of DA is contacted with the test strip, and the test strip is inserted into the reflectometer, whereby the color is determined by the reflectometer, and the instrument reports a numeric value proportional to the amount of DA in the test sample.

In another embodiment, the medical device detects electrons by electrochemical means.

In another embodiment, the invention provides methods for the diagnosis of vaginitis resulting from fungal organisms.

In another embodiment, the invention provides methods for detecting DA on inanimate objects and/or matrices comprising contacting the sample with a DAOx enzyme in the presence of oxygen and detecting hydrogen peroxide using colorimetric or electrochemical means. Alternatively, the sample can be an extract from the inanimate object or matrix using aqueous or organic solvents. In one embodiment, the inanimate object is a catheter or other object inserted in the body. Other examples of inanimate objects include, but are not limited to, prosthetic devices, orthopedic devices, surgical tools and devices, stents, ventilator tubes, electrodes, screws, wires, etc.

In another embodiment, detection of fungi on inanimate objects is by detecting electrons using calorimetric or electrochemical means. Alternatively, the sample can be an extract from the inanimate object or matrix using aqueous or organic solvents.

Other embodiments include the use of the detection of DA using a DAOx enzyme to determine which drug to use in treating a fungal infection, monitoring of drug efficacy in the treatment of a fungal infection, and the determination of the seriousness of a fungal infectious disease (i.e., disease staging).

### EXAMPLES

### Example 1

### Identification of Genes with Putative D-Arabinitol Oxidase Activity

An enzyme with oxidoreductase activity toward sorbitol and xylitol has been previously purified from an unknown species of the actinomycete genus Streptomyces (Oda and Hiraga, 1998) (SEQ ID NO:6). Prior characterization of this sorbitol oxidase revealed that it also catalyzed the oxidation of D-arabinitol. When this protein was used in the methods of the instant invention as a query in a blast search of the GenBank non-redundant protein sequence database (nr) it demonstrated a high degree of similarity with predicted proteins encoded in the complete genome sequences of Streptomyces coelicolor A3(2) (SEQ ID NO:2) (60% identity, blastp expect value=1×10⁻¹²⁸) and Streptomyces avermitilis MA-4680 (SEQ ID NO:4) (56% identity, blastp expect value=1×10⁻¹²⁰) (Bentley et al., 2002; Ikeda et al., 2003). The S. coelicolor homolog (ScOx) is 1,257 nucleotides long and is predicted to encode a 418 amino acid protein with a molecular weight of 44.3 Kd (SEQ ID NO:2). The S. avermitilis homolog (SaOx) is 1,269 nucleotides long and is predicted to encode a 422 amino acid protein with a molecular weight of 44.9 kD (SEQ ID NO:4).

### Example 2

### Cloning and Expression of DA Oxidases from Streptomyces coelicolor and Streptomyces avermitilis

The foregoing ScOx and SaOx genes are isolated via PCR amplification from the corresponding genomic DNA (American Type Culture Collection, ATCC). The PCR primers used for this amplification enable cloning and expression of three expression derivatives for each source of DNA; a N-terminal histidine tagged form, a C-terminal histidine tagged form, and the native untagged form of the protein (Table 1). The histidine fusion tags facilitate protein detection and purification while the untagged proteins are used to produce non-tagged native proteins. The PCR products are cloned into plasmid pET-30a for expression (Novagen).

TABLE 1

PCR Primers for Recovery of ScOx and SaOx

| | | |
|---|---|---|
| Name | Description | Sequence* |

| | | |
|---|---|---|
| 5_Nhis_ScOx | 5′ primer for protein | GgtccatggctAGC |
| | recovery from S. | GACATCACGGT |
| | coelicolor and intro- | CACC |
| | duction of a N-termi- | |
| | nal six-histidine | |
| | tag. | |
| 5_NoTag_ScOx | 5′ primer for protein | ggccatATGAGCG |
| | recovery from S. | ACATCACGGTC |
| | coelicolor without a | ACC |
| | N-terminal six-histi- | |
| | dine tag. | |
| 3_Chis_ScOx | 3′ primer for protein | ggcctcgagGCCC |
| | recovery from S. | GCGAGCACCCC |
| | coelicolor and intro- | GC |
| | duction of a C-termi- | |
| | nal six-histidine | |
| | tag. | |
| 3_NoTag_ScOx | 3′ primer for protein | ggcctcgagTCAGC |
| | recovery from S. | CCGCGAGCACC |
| | coelicolor without a | CCGC |
| | C-terminal six-histi- | |
| | dine tag. | |
| 5_Nhis_SaOx | 5′ primer for protein | ggtccatggctACTG |
| | recovery from S. | ACCCAGGGACC |
| | avermitilis and in- | GC |
| | troduction of a N- | |
| | terminal six-histi- | |
| | dine tag. | |
| 5_NoTag_SaOx | 5′ primer for protein | ggccatATGACTG |
| | recovery from S. | ACGCAGGGACC |
| | avermitilis without a | GC |
| | N-terminal six-histi- | |
| | dine tag. | |
| 3_Chis_SaOx | 3′ primer for protein | ggcctcgagCGAC |
| | recovery from S. | GGCCGGTCGCC |
| | avermitilis and in- | GG |
| | troduction of a C- | |
| | terminal six-histi- | |
| | dine tag. | |
| 3_NoTag_SaOx | 3′ primer for protein | ggcctcgagTCACG |
| | recovery from S. | ACGGCCGGTCG |
| | avermitilis without a | CCGG |
| | C-terminal six-histi- | |
| | dine tag. | |

*Nucleotides in the oxidase protein coding regions are in uppercase. Restriction end nuclease cleavage sites are underlined. The restriction enzymes and their recognition sites to be used are NdeI (catatg), NcoI (ccatgg), and XhoI (ctcgag).

The expression vectors are used to transform the Rosetta (DE3/pLysS) strain of E. coli. Protein expression is accomplished by the growth of transformed bacterial cultures in the presence of IPTG (isopropyl β-D-1-thiogalactopyranoside) at 23° C. for 16-18 hours. Cells are harvested by centrifugation and protein extracts are prepared by resuspending the cells in one-tenth initial culture volume of BugBuster (primary amine free, Novagen) containing 12.5 U/ml benzonase (Novagen). Extracts are used directly in protein activity assays as described in Example 3. DAOx proteins containing histidine-tags (His-tags) are purified further as described in the following paragraph.

The histidine tagged proteins in extracts are recovered via affinity chromatography using Ni-agarose resin (Pierce) as follows. 10 ml E. coli culture containing the recombinant plasmid is grown at 23° C. for 16-18 hours. The cells are recovered by centrifugation, and lysed in 1.0 ml BugBuster (primary amine free, Novagen) containing 12.5 U/ml benzonase (Novagen) for 5 minutes at room temperature. 100 μl Ni-Agarose affinity resin (Pierce) is placed into a polypropylene plastic column and washed with 1.0 ml 20 mM Tris (pH=8). The cell extract is allowed to flow through the washed Ni-Agarose resin, collected, and allowed to flow over the resin a second time. The resin is then washed with 1.0 ml wash buffer (20 mM Tris (pH=8), 300 mM NaCl, 20 mM imidazole). The DAOx His-tag proteins are eluted in 200 μl elution buffer (20 mM Tris (pH=8), 300 mM NaCl, 250 mM imidazole), to recover 200 μl Ni-affinity purified protein. The Ni-affinity purified protein is desalted using Zeba 0.5 ml desalt columns (Pierce) using 2 columns for each extract as follows. Two desalt columns are centrifuged in a 1.5 ml microfuge tube at 2000 rpm for 1 minute, pre-equilibrated with 0.5 ml buffer containing 200 μl 20 mM Tris (pH=8) 100 mM NaCl, and centrifuged in a 1.5 ml microfuge tube at 2000 rpm for 1 minute. 100 μl Ni-affinity purified protein preparation is applied to each desalt column and the column is centrifuged in a 1.5 ml microfuge tube at 2000 rpm for 1 minute. The purified and desalted DAOx protein is recovered in the combined eluate from the two columns. Bradford assays are used to compare purified proteins with standard protein concentrations to determine the DAOX protein concentrations. Protein purity is examined via SDS-PAGE electrophoresis using methods well known in the art. The final purified proteins are stable and are stored at 4° C., or made to 50% glycerol and stored at −20° C.

### Example 3

### Determination of Enzyme Activity

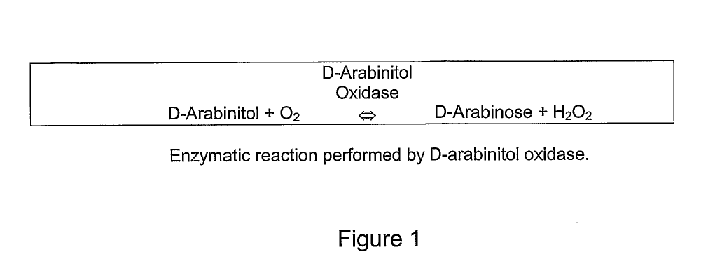

The D-arabinitol oxidase enzyme activity is assayed by the formation of H₂O₂ during the enzyme reaction (FIG. 1) as coupled with a peroxidase indicator reaction that catalyzes the oxidation of 3,3′,5,5′-tetramethylbenzidine hydrochloride (TMB), producing a colored product. An increase in absorbance is a direct measure of D-arabinitol oxidase activity. Enzyme activity is assayed in crude extracts and in purified protein preparations. One to 10 μl of DAOx enzyme in solution (extract, or Ni-affinity purified and desalted) is assayed in a final volume of 100 μl assay buffer containing 20 mM potassium phosphate (pH=6.5), 1 mM 3,3′,5,5′-tetramethylbenzidine hydrochloride (TMB), 10 mM sugar alcohol, 1.25 U/ml of horse radish peroxidase (Sigma), and 10 mM sugar alcohol substrate in a microtiter plate well. In this reaction, formation of H₂O₂ results in the formation of blue color measurable by reading the absorbance at 650 nanometers (nm). Absorbance at 650 nm is followed kinetically using a Tecan Rainbow spectrophotometer at room temperature (23° C.). Milli-absorbance units per minute (mA/min) are calculated as the increase in absorbance at 650 nm/minute×1000. It is recognized that researchers skilled in the art may perform the assay as an end-point assay, by stopping reactions at various times with a solution of acid to produce a yellow color, and reading the absorbance at 450 nm. It is also recognized that those skilled in the art can determine the optimal conditions for DAOx proteins and their mutant variants for temperature, pH, and ionic strength by assaying for oxidase activity in similar reaction conditions by varying the temperature, salts concentrations, and pH of the reaction conditions using methods well known to researchers in the art. It is also recognized that the biochemical characteristics for DAOx proteins and mutant variants, such as Kₘ, Vₘₐₓ, and k_{cat} for preferred and non-preferred substrates such as arabinitol, xylitol, sorbitol, mannitol, ribitol, galactitol, and i-erythritol in both D- and L-forms as available, can be determined for each enzyme based on kinetic experiments at various sugar alcohol substrate concentrations (typically between 0.1 nM to 100 mM) using methodology well known to researchers in the art.

### Example 4

### Engineering of Optimized Proteins by Error-Prone PCR

DNA sequences encoding an SaOx protein (SEQ ID NO:3) are used as starting material for error prone PCR. Error-prone PCR is accomplished by performing PCR from DNA templates using a non-proof reading DNA polymerase such as Taq Polymerase, or by using a GeneMorph II Random Mutagenesis Kit (Stratagene). For mutagenesis using Taq Polymerase, 10 ng of genomic DNA from Streptomyces avermitilis MA-4680 (ATCC) is amplified using buffer conditions of 1×PCR Buffer II (Roche), 2 mM MgCl₂, 0.2 mM dNTP mix, 5% DMSO, 20 pmoles each of the primers, 5′-ggccatatgactgacgcagggaccgc, and 5′-ggcctcgagcgacggccggtcgccgg, and 5 units Taq polymerase (Roche). PCR is performed in a thermocycler as 99° C. for 3 minutes, 95° C. for 3 minutes, 30 cycles of [95° C. 30 for seconds, 72° C. for 1.5 minutes], 72° C. for 5 minutes, and a final hold temperature of 20° C. The same primers are used for error prone PCR using the GeneMorph II kit following the instructions provided by the manufacturer with the following modification, DMSO is added to the reaction mix to a final concentration of 5%. The error prone PCR products are isolated by gel electrophoresis, purified using a QiaQuick gel extraction kit (Qiagen), cut with restriction enzymes NdeI and XhoI, and cloned into plasmid pET-30a that has also been cut with NdeI and XhoI and gel isolated and purified. Mutant proteins are expressed in an E. coli strain, protein extracts are prepared, and DAOx enzyme assays are performed with substrates, D-sorbitol and D-arabinitol, as described in Example 3. Variants showing greater activity against DA, similar activity against DA and less activity against other sugar alcohols, or greater activity against DA and less activity against other sugar alcohols are identified. Table 2 shows examples of mutants identified after error prone PCR as having a change in the sorbitol/DA.

TABLE 2
Improved mutants identified after error prone PCR

| | | Amino acid changes |
|---|---|---|
| Mutant | Sorb/DA | from SEQ ID NO:4 |
| SaOX (wild type + His-tag) | 14.8 | STOP423LEHHHHHH |
| | | (C-terminal His-tag) |
| SaOX-pTeo020 | 6.6 | W11 seC* I213T V252M |
| | | (+C-terminal His-tag) |
| SaOX-B6A7 | 5.0 | W11 seC* I213T A234T |
| | | V252M (+C-terminal His-tag) |
| SaOX-B5B5 | 5.4 | W11 seC* A77T I213T V252M |
| | | (+C-terminal His-tag) |

SaOX, Streptomyces avermitills oxidase, SaOX-pTeo020, mutant obtained from error-prone PCR using genomic Streptomyces avermitilis oxidase as template with Taq polymerase.
SaOX-B6A7 and -B5B5, mutants obtained from error-prone PCR using SaOX-pTeo020 as template with GeneMorph II kit.
Changes in protein coding region: single letter amino acid code, position, change.
DA activity measured as described in text.
Sorb/DA, ratio of oxidase activity with sorbitol as substrate to DA as substrate.
seC*, selenocysteine.

### Example 5

### Detection of DA in Mammalian Serum Using Diagnostic Test Strips

An aliquot of human serum is placed onto a strip consisting of a solid polymer backing, absorbent matrix and immobilized reagents including a DA oxidase. The strip is inserted into a reflectometer which measures the color development of the strip. The color development of negative controls (reagents with no serum, and serum with no reagents) is subtracted from the value obtained with reagents and serum together. Positive controls are derived from regions of test strips containing known quantities of DA. The amount of DA present in the serum sample is determined by comparing the reluctance value obtained with reagents and serum together with the reflectance value obtained for the positive controls.

### Example 6

### Detection of DA in Mammalian Serum Using a Diagnostic Kit

A 1.0 ml aliquot of human serum is pre-warmed to 37° C. in a sterile tube. A permeable polymer tablet containing immobilized reagents including a DA oxidase is added to the tube, and the tube is incubated with gentle agitation for 20 minutes. The color development in the serum is determined by measurement of absorbance using a spectrophotometer. The color development of negative controls (tablet with no serum, and serum with no tablet) is subtracted from the value obtained with tablet and serum together. Positive controls are derived from the addition of tablets to solutions of known quantities of DA. The amount of DA present in the serum sample is determined by comparing the absorbance value obtained with tablet and serum together with the absorbance values obtained the positive controls.

While the foregoing describes certain embodiments of the invention, it will be understood by those skilled in the art that variations and modifications may be made and still fall within the scope of the invention. The foregoing examples are intended to exemplify various specific embodiments of the invention and do not limit its scope in any manner.

What is claimed is:

1. A method of detecting the presence of D-arabinitol, which was produced by a fungus, in a test sample comprising:
a) obtaining a test sample;
b) selecting a predetermined amount of an enzyme having D-arabinitol oxidase activity;
c) contacting the test sample with the enzyme in the presence of oxygen;
d) measuring the amount of hydrogen peroxide produced by the reaction of the test sample and the enzyme; and
e) determining if the amount of hydrogen peroxide produced is sufficient to indicate the presence of a D-arabinitol produced by a fungus.

2. A method according to claim 1 wherein the amount of hydrogen peroxide produced is measured by a calorimetric method.

3. A method according to claim 1 wherein the amount of hydrogen peroxide produced is measured by an electrochemical method.

4. A kit of parts suitable for the detection of the presence of D-arabinitol, produced by a fungus, in a test sample comprising: an enzyme having D-arabinitol oxidase activity capable of oxidizing D-arabinitol in the presence of oxygen to produce hydrogen peroxide and a means for detecting the amount of hydrogen peroxide produced by the reaction of D-arabinitol and the enzyme.

5. A kit of parts according to claim 4 wherein the kit is a test strip comprising the enzyme and a calorimetric indicator for hydrogen peroxide.

6. A method of producing an enzyme which preferentially oxidizes D-arabinitol in the presence of oxygen to produce hydrogen peroxide over oxidizing other sugar alcohols comprising:
a) producing at least one mutation in a first gene encoding a first enzyme having D-arabinitol oxidase activity to produce a second gene encoding a second enzyme having D-arabinitol oxidase activity;
b) measuring the enzyme activity, produced by the second enzyme, to oxidize D-arabinitol versus its ability to oxidize at least one other sugar alcohol;
c) calculating the ratio of the D-arabinitol oxidase activity to the oxidase activity with at least one other sugar alcohol for the second enzyme;
d) comparing the calculated ratio for the second enzyme to the same ratio for the first enzyme;
e) selecting the second enzyme if the second enzyme has a higher ratio than the first enzyme.


SEQUENCE LISTING (8 sequences)

SEQ ID NO: 1 - DNA, 1257 bp, Streptomyces coelicolor
atgagcgacatcacggtcaccaactgggccggcaacatcacgtacacggcgaaggaactgctgcggccgcactccctggacgcgctgcgggccctggtggcggacagcgccagggtgcgggtgctgggcagcgggcactccttcaacgagatcgccgagccgggcgacgggggtgtcctgctgtcgctggcgggcctgccgtccgtggtggacgtggacacggcggcccgtacggtgcgggtcggcggcggtgtgcggtacgcggagctggcccgggtggtgcacgcgcggggcctggcgctgccgaacatggcctcgctgccgcacatctcggtcgccgggtcggtggccaccggcacccacggttcgggggtgggcaacggttcgctggcctcggtggtgcgcgaggtggagctggtcaccgcggacggttcgaccgtggtgatcgcgcggggcgacgagcggttcggcggggcggtgacctcgctcggcgcgctgggcgtggtgacgtcgctcacactcgacctggagccggcgtacgagatggaacagcacgtcttcaccgagctgccgctggccgggttggacccggcgacgttcgagacggtgatggcggcggcgtacagcgtgagtctgttcaccgactggcgggcgcccggtttccggcaggtgtggctgaagcggcgcaccgaccggccgctggacggtttcccgtacgcggccccggccgccgagaagatgcatccggtgccgggcatgcccgcggtgaactgcacggagcagttcggggtgccggggccctggcacgagcggctgccgcacttccgcgcggagttcacgcccagcagcggtgccgagttgcagtcggagtacctgatgccccgggagcacgccctggccgccctgcacgcgatggacgcgatacgggagacgctcgcgccggtgctccagacctgcgagatccgcacggtcgccgccgacgcgcagtggctgagcccggcgtacgggcgggacaccgtggccgcgcacttcacctgggtcgaggacacggcggcggtgctgccggtggtgcggcggctggaggaggcgctcgtccccttcgcggcccgtccgcactgggggaaggtgttcaccgtcccggcgggcgagctgcgtgcgctgtacccgcggctggccgacttcggggcgctggccggggcgctggacccggcggggaagttcaccaacgcgttcgtgcgcggggtgctcgcgggctga

SEQ ID NO: 2 - protein, 418 aa, Streptomyces coelicolor
MSDITVTNWAGNITYTAKELLRPHSLDALRALVADSARVRVLGSGHSFNEIAEPGDGGVLLSLAGLPSVVDVDTAARTVRVGGGVRYAELARVVHARGLALPNMASLPHISVAGSVATGTHGSGVGNGSLASVVREVELVTADGSTVVIARGDERFGGAVTSLGALGVVTSLTLDLEPAYEMEQHVFTELPLAGLDPATFETVMAAAYSVSLFTDWRAPGFRQVWLKRRTDRPLDGFPYAAPAAEKMHPVPGMPAVNCTEQFGVPGPWHERLPHFRAEFTPSSGAELQSEYLMPREHALAALHAMDAIRETLAPVLQTCEIRTVAADAQWLSPAYGRDTVAAHFTWVEDTAAVLPVVRRLEEALVPFAARPHWGKVFTVPAGELRALYPRLADFGALAGALDPAGKFTNAFVRGVLAG

SEQ ID NO: 3 - DNA, 1269 bp, Streptomyces avermitilis
atgactgacgcagggaccgcgctcaccaactgggccgggaacatcacgtactcggccaaggagctgcaccggccgcaatcgctggacgcgctgcgggcgctggtcgcggacagtgcgaaggtgcgggtactgggcagcgggcactcgttcaacgagatcgccgagccgggtgccgacggcgtactgctgtcgctgaccgccctgccgccgtcggtcgaggtggacacggccgcccgtaccgtacgggtcgcgggcggggtccggtacgccgaactcgcccgggtggtgcacggacacgggctcgcgctgcccaacatggcctcgctcccgcacatctccgtggcgggctcggtggccaccggcacgcatggctccggcgtcaccaacggctcgctcgcctcggccgtgcgggaggtggagctggtcacggccgacggttcggcggtgcggatcgggcggggcgacgaccggttcgacggcgccgtgaccgcgctcggggcgctcggggtggtcaccgcgctcacgctcgatctggagccggactaccgggtcgcccagcaggttttcaccgaactcccgctggcggggctggacttcgatgcggtggcggcttcggcgtacagcgtcagcctcttcaccggctggcggacgtcgggctttgcgcaggtgtggctcaagcgccgcaccgaccggccgtcggccgacttcccgtgggcggcgccggccaccgaggcgatgcaccccgtgccgggcatgcccgcggtcaactgcacccagcagttcggggtcccgggcccctggcacgagcgactcccgcacttccgggcggagttcacccccagcagcggcgccgagctccagtcggagtacctgctgccgcgcccgtacgccctcgacgcgctgcacgccctggacgccgtgcgggagacggtggcgccggtgctccagatctgcgaggtgcggacggtcgccgccgacgcgcagtggctgagccccgcctacgggcgggacaccgtggcgctgcacttcacctgggtcgaggacctggccgccgtactgcccgtggtgcggcgggtcgaggaggcgctggaccccttcgacccgcggccgcactggggcaaggtcttcgcggtcccggcgcgggtgctgcgcgggcggtatccgcgactgggcgatttccgggccctggtggactcgctcgatcccggcggaaagttcaccaacgccttcgtacgggaggtgctgggctccggcgaccggccgtcgtga

SEQ ID NO: 4 - protein, 422 aa, Streptomyces avermitilis
MTDAGTALTNWAGNITYSAKELHRPQSLDALRALVADSAKVRVLGSGHSFNEIAEPGADGVLLSLTALPPSVEVDTAARTVRVAGGVRYAELARVVHGHGLALPNMASLPHISVAGSVATGTHGSGVTNGSLASAVREVELVTADGSAVRIGRGDDRFDGAVTALGALGVVTALTLDLEPDYRVAQQVFTELPLAGLDFDAVAASAYSVSLFTGWRTSGFAQVWLKRRTDRPSADFPWAAPATEAMHPVPGMPAVNCTQQFGVPGPWHERLPHFRAEFTPSSGAELQSEYLLPRPYALDALHALDAVRETVAPVLQICEVRTVAADAQWLSPAYGRDTVALHFTWVEDLAAVLPVVRRVEEALDPFDPRPHWGKVFAVPARVLRGRYPRLGDFRALVDSLDPGGKFTNAFVREVLGSGDRPS

SEQ ID NO: 5 - DNA, 1263 bp, Streptomyces
atgacccccgcggagaagaactgggccggcaacatcaccttcggcgcgaagcggctgtgtgtgccgagatccgtccgggaactgcgcgagacggtcgccgcctccggcgcggtccgccccctggggacccggcactccttcaacaccgtcgcggacacctccggcgaccatgtgtcgctcgccggactcccgcgcgtcgtggacatcgacgtcccgggccgcgccgtctcgctgtccgccggactccgcttcggggagttcgccgccgaactgcacgcgcgcggtctcgccctcgccaacctgggctcgctcccgcacatctcggtcgccggcgccgtcgcgaccggcacccacggctcgggcgtgggcaaccgctcattggcgggcgcggtgcgtgccctctccctggtgacggccgacggggagacgcgcaccctgcggcgcaccgacgaggacttcgcgggcgcggtcgtctccctcggcgccctcggcgtggtgacgtcgctggaactcgacctcgtgcccgccttcgaggtgcgccagtgggtgtacgaggacctgcccgaggcgacactcgccgcgcgcttcgacgaggtgatgtccgccgcctacagcgtcagcgtcttcaccgactggcgccccggcccggtcggccaggtctggctcaagcagcgggtgggcgacgagggggcacggtccgtgatgcccgccgagtggctgggcgcccggctcgccgacggcccccggcacccggtccccgggatgcccgccgggaactgcacggcgcagcagggggtgcccgggccctggcacgagcggcttccgcacttccggatggagttcaccccgagcaacggcgacgagctccagtcggagtacttcgtggcccgcgcggacgccgtcgccgcctacgaggccctggcccggctgcgggaccggatcgccccggtgctccaggtctccgagctccgcaccgtcgccgccgacgacctgtggctgagccccgcccacggccgggactcggtggccttccacttcacctgggtcccggacgcggcggccgtcgcgccggtggccggggcgatcgaggaggccctggccccgttcggcgcccgcccgcactggggcaaggtcttctcgacggcccccgaggtgctgcgcacgctctacccgcgctacgcggacttcgaggagctggtgggccgccacgaccccgagggcaccttccgcaacgcgttcctggaccggtacttccgccgctga

SEQ ID NO: 6 - protein, 420 aa, Streptomyces
MTPAEKNWAGNITFGAKRLCVPRSVRELRETVAASGAVRPLGTRHSFNTVADTSGDHVSLAGLPRVVDIDVPGRAVSLSAGLRFGEFAAELHARGLALANLGSLPHISVAGAVATGTHGSGVGNRSLAGAVRALSLVTADGETRTLRRTDEDFAGAVVSLGALGVVTSLELDLVPAFEVRQWVYEDLPEATLAARFDEVMSAAYSVSVFTDWRPGPVGQVWLKQRVGDEGARSVMPAEWLGARLADGPRHPVPGMPAGNCTAQQGVPGPWHERLPHFRMEFTPSNGDELQSEYFVARADAVAAYEALARLRDRIAPVLQVSELRTVAADDLWLSPAHGRDSVAFHFTWVPDAAAVAPVAGAIEEALAPFGARPHWGKVFSTAPEVLRTLYPRYADFEELVGRHDPEGTFRNAFLDRYFRR

SEQ ID NO: 7 - DNA, 1248 bp, streptomyces
atgagcacagccgtgaccaactgggcggggaacatcacctacaccgccaaggaggtgcaccggccggccaccgccgaggaactggcggacgtggtcgcgcgcagtgcatggggtgcgtgtgctggggcagcgggccactcgttcaacgagatcgccgacccgggtcccgacggcgtcctgctgcgcctggacgcgctgcccgccgagaccgacgtggacaccacggcccgcacggtccgggtcggcggcggtgtccgctacgccgaactggcccgcgtggtgcacgcccacggactggccctgcccaacatggcgtcgctgccgcacatctcggtggccgggtcggtggccaccggcacccacggctcgggggtgaccaacggtcccctggcggcaccggtgcgcgaggtggagctggtcacggcggacggctcgcaggtacgcatagccccgggcgaacgccgcttcggcggggcggtcacctccctcggcgcgctcggcgtcgtcaccgcgctcaccctggacctggagcccgccttcgaggtggggcagcatctgttcacggagctgccgctgcgagggctggacttcgagacggtcgccgccgccgggtacagcgtcagcctgttcaccgactggcgggagcccggcttccggcaggtgtggctcaagcggcgcaccgaccaggagctgcccgacttcccgtgggcgcggccggcgaccgtggcgctgcacccggtgcccgggatgcccgcggagaactgcacgcagcagttcggggtgccgggcccctggcacgagcggctgccgcacttccgggcggagttcaccccgagcagcggtgccgaactccagtccgagtacctgctgccgcgtgcccacgccctcgacgcgctggacgccgtggaccggatccgggacaccgtcgcgcccgtgctgcagacctgcgaggtccgcaccgtcgcccccgacgagcagtggctcggcccgagccacggccgggacaccgtggccctgcacttcacctgggtgaaggacaccgaggcggtgctgccggtggtgcggcgcctggaggaggcgctggacgccttcgacccgcgcccccactggggcaaggtgttcacgacgtccgccgccgccctgcgcgcccggtacccgcggctggcggacttccgggccctggcccgcgagctggacccgtcggggaagttcaccaacaccttcctgcgggacctgctggacggctga

SEQ ID NO: 8 - protein, 415 aa, Streptomyces
MSTAVTNWAGNITYTAKEVHRPATAEELADVVARSAWGACAGAAGHSFNEIADPGPDGVLLRLDALPAETDVDTTARTVRVGGGVRYAELARVVHAHGLALPNMASLPHISVAGSVATGTHGSGVTNGPLAAPVREVELVTADGSQVRIAPGERRFGGAVTSLGALGVVTALTLDLEPAFEVGQHLFTELPLRGLDFETVAAAGYSVSLFTDWREPGFRQVWLKRRTDQELPDFPWARPATVALHPVPGMPAENCTQQFGVPGPWHERLPHFRAEFTPSSGAELQSEYLLPRAHALDALDAVDRIRDTVAPVLQTCEVRTVAPDEQWLGPSHGRDTVALHFTWVKDTEAVLPVVRRLEEALDAFDPRPHWGKVFTTSAAALRARYPRLADFRALARELDPSGKFTNTFLRDLLDG